(19) United States
(12) Patent Application Publication
(10) Pub. No.: US 2016/0296289 A1
(43) Pub. Date: Oct. 13, 2016

(54) CUSTOM MATCHED JOINT PROSTHESIS REPLACEMENT

(71) Applicant: CONCEPTO LLC, Cincinnati, OH (US)

(72) Inventors: Sambhu Choudhury, Cincinnati, OH (US); Sean Lynch, Cincinnati, OH (US); Arturo David Sanchez, Cincinnati, OH (US); Andrew Ryan Law, Cincinnati, OH (US)

(21) Appl. No.: 15/186,853

(22) Filed: Jun. 20, 2016

Related U.S. Application Data
(63) Continuation-in-part of application No. 14/216,798, filed on Mar. 17, 2014, now abandoned.
(60) Provisional application No. 61/798,742, filed on Mar. 15, 2013.
(60) Provisional application No. 61/813,836, filed on Apr. 19, 2013.

(51) Int. Cl. A61B 34/10; B33Y 10/00; G05B 15/02; B33Y 50/02; B29C 67/00; B22D 11/01; A61F 2/34; B33Y 30/00

(52) U.S. Cl. CPC A61B 34/10; A61F 2/34; B33Y 10/00; B33Y 30/00; B33Y 50/02; B29C 67/0088; B29C 67/0059; B22D 11/01; G05B 15/02; A61B 2034/108; A61F 2310/00029; A61F 2002/3479; C22C 19/07

(57) ABSTRACT

An apparatus and method of fabricating a replacement prosthesis component for implantation into a patient by receiving a diagnostic scan of an implanted prosthesis component in the patient. A controller converts the diagnostic scan into a three-dimensional model of the implanted prosthesis. The controller automatically matches the three-dimensional model with a selected replacement part model that mates with the implanted prosthesis. The controller prepares a three-dimensional printing model of the selected replacement part model to a three-dimensional printer for fabricating a matching replacement part.

### CLAIM OF PRIORITY UNDER 35 U.S.C. §120

The present Application for Patent is a continuation-in-part of patent application Ser. No. 14/216,798 entitled “Inner Acetabular Liner for a Dual Mobility Femoral Head Construct” filed 17 Mar. 2014, pending, which in turn claimed priority to U.S. Provisional Application No. 61/798,742 filed 15 Mar. 2013 and or U.S. Provisional Application No. 61/813,836 filed 19 Apr. 2013, all assigned to the assignee hereof and hereby expressly incorporated by reference herein.

### FIELD OF THE INVENTION

The present disclosure generally relates to prosthetic medical devices, and more particularly to cementable cast CoCr metal liners in conjunction with dual mobility femoral head constructs, and more particularly to a method of ascertaining and custom fabricating the matching replacement prosthesis.

### BACKGROUND OF THE INVENTION

Total hip replacement surgery is commonly performed to alleviate pain and loss of function in injured and diseased hip joints. During this surgery, the articulating surfaces of the hip joint are replaced with prosthetic bearing components. The replacement components generally include a femoral component having a convex bearing surface and an acetabular cup component having a mating concave bearing surface.

Modular femoral and acetabular components have become popular because they allow the surgeon to assemble components in a variety of configurations at the time of surgery to meet specific patient needs relative to size and geometry. For example, modular femoral components generally include separate stem and head components that can be assembled in a variety of configurations of surface finish, stem diameter, stem length, proximal stem geometry, head diameter, and neck length. Likewise, modular acetabular components generally include separate shell and liner components that can be assembled in a variety of configurations of surface finish, shell outer diameter, liner inner diameter, and constraining fit with the femoral head.

A common clinical scenario encountered by an orthopedic surgeon is a patient with a secure cementless acetabular shell and a failed articular head insert due to failed wear properties, or instability. One treatment option is to cement a new liner into the fixed shell. This is an optimal treatment option as it retains the existing acetabular shell component without compromising existing acetabular bone stock. Unfortunately, stability is dictated by the market's current liner options, and inner diameter head options. In order to optimize stability, the outer diameter liner, with the largest inner diameter head acceptance would be cemented into the existing acetabular component. A necessary cement mantle of 0.5 mm is needed to ensure stability of the liner into the acetabular component.

### SUMMARY OF THE INVENTION

In one aspect, the present disclosure provides a method of fabricating a replacement prosthesis component for implantation into a patient. In one or more embodiments, the method includes receiving a diagnostic scan of an implanted prosthesis component in the patient. The method includes converting the diagnostic scan into a three-dimensional model of the implanted prosthesis. The method includes automatically matching the three-dimensional model with a selected replacement part model that mates with the implanted prosthesis. The method includes preparing a three-dimensional printing model of the selected replacement part model to a three-dimensional printer for fabricating a matching replacement part.

In another aspect, the present disclosure provides an apparatus of fabricating a replacement prosthesis component for implantation into a patient. In one or more embodiments, the apparatus includes a memory containing three-dimensional information on more than one type of replacement prosthesis component. The apparatus includes a three-dimensional printer and a controller communicatively coupled to the memory and the three-dimensional printer. The controller (i) receives a diagnostic scan of an implanted prosthesis component in the patient; (ii) converts the diagnostic scan into a three-dimensional model of the implanted prosthesis; (iii) automatically matches the three-dimensional model with a selected replacement part model that mates with the implanted prosthesis; and (iv) prepares a three-dimensional printing model of the selected replacement part model to the three-dimensional printer for fabricating a matching replacement part.

These and other features are explained more fully in the embodiments illustrated below. It should be understood that in general the features of one embodiment also may be used in combination with features of another embodiment and that the embodiments are not intended to limit the scope of the invention.

### BRIEF DESCRIPTION OF THE DRAWINGS

The various exemplary embodiments of the present invention, which will become more apparent as the description proceeds, are described in the following detailed description in conjunction with the accompanying drawings, in which:

FIG. 1 illustrates a diagram of an isometric, exploded view of a dual mobility femoral head construct according to one or more embodiments.

FIG. 2 illustrates a front view in vertical cross section of the dual mobility femoral head construct implanted into a pelvis and femur.

FIG. 3 illustrates an isometric, top view of an inner acetabular shell for the dual mobility femoral head construct of FIG. 1 according to one version.

FIG. 4 illustrates a top view of the inner acetabular shell of FIG. 3;

FIG. 5 illustrates a side view of the inner acetabular shell of FIG. 3;

FIG. 6 illustrates a side cross section view of the inner acetabular shell of FIG. 5 taken along lines A-A;

FIG. 7 illustrates a detail view within circle B of the inner acetabular shell of FIG. 6, according to one or more embodiments;

FIG. 8 illustrates a block diagram of medical scanning and fabrication system, according to one or more embodiments; and

FIG. 9 illustrates a flow diagram of a method of medically scanning and fabricating a custom replacement of a joint prosthesis replacement component, according to one or more embodiments.

### DETAILED DESCRIPTION

The present invention relates to a prosthetic acetabular liner for replacing the natural bearing surface of the hip acetabulum. A standard total hip prosthesis comprises two parts constituting a ball-and-socket joint, namely a first part intended to be implanted in the pelvis of a patient, and a second part intended to be implanted in the femur.

The first part of the prosthesis generally has a stem which is intended to engage in the medullary canal of the femur, and of which the proximal end is connected by a neck to a spherical head, or ball, intended to engage in the female part, or socket, of the joint.

The second part of the prosthesis, which has to be implanted in the pelvis, and which will be designated generally by the word acetabulum, normally comprises a hemispherical insertion cup, which is placed in a prepared cotyloid cavity of the pelvic bone, and in which is placed an articular insert, which is designed to receive the spherical head. The insertion cup is commonly made of metal. The articular insert is made of a material with a low coefficient of friction, such as polyethylene or a ceramic.

In one aspect, the present disclosure provides an implantable prosthetic device having a truncated hemispherical liner (165 degrees) formed from a cast cobalt-chromium alloy and having an outer diameter sized for attachment inside a secure acetabular shell implanted into acetabular components recessed in a pelvis. At least three spacers are annularly displaced about an outer diameter of the liner to define a uniform cement thickness within the secure acetabular shell. Web shaped depressions are formed circumferentially and longitudinally in the outer diameter of the hemispherical liner to receive cement. This webbing is designed to resist both torsional and lever-out forces of hip kinematics.

In another aspect, the present disclosure provides the acceptance of a dual mobility femoral head construct being received into the liner, within an acetabular component recessed in a pelvis. An implantable prosthetic device has a truncated hemispherical liner formed from a cast cobalt-chromium alloy and sized for cement bonding inside the secure acetabular shell. At least three spacers annularly are displaced about an outer diameter of the truncated hemispherical liner to define a uniform cement thickness within the secure acetabular shell. Web shaped depressions are formed circumferentially and longitudinally in the outer diameter of the hemispherical liner to receive cement. This webbing is designed to resist both torsional and lever-out forces of hip kinematics.

The dual mobility head construct articulates within the inner diameter of the truncated hemispherical liner. The construct is comprised of a modular femoral head that is fixed onto a femoral prosthetic neck, which then articulates into a larger diameter modular polyethylene head. This construct optimizes wear at the modular femoral head/polyethylene interface, and optimizes stability at the polyethylene/cast CoCr liner interface. Optimizing the largest polyethylene head diameter optimizes hip stability.

Thus in one or more embodiments, dual mobility femoral head construct has a secure acetabular shell received within an acetabular recess formed in a pelvis. An implantable prosthetic device is implantable after a failure of an originally installed articular head insert of an acetabular cup assembly. A hemispherical liner is formed from a cast cobalt-chromium alloy for thinness and elasticity. At least three spacers are annularly displaced about an outer diameter of the hemispherical liner to define a uniform cement thickness with the secure acetabular shell. Web shaped depressions are formed circumferentially in the outer diameter of the hemispherical liner to receive cement to resist lever out forces and to secure the cement bond with the secure acetabular shell. A replacement articular head insert is then received for dual mobility rotational movement in an inner diameter of the hemispherical liner. A femoral head implant is received for articulating movement in the articular head insert.

Turning now to the Drawings, wherein like reference numerals refer to like components throughout the several views. The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well-known structures and components are shown in block diagram form in order to avoid obscuring such concepts.

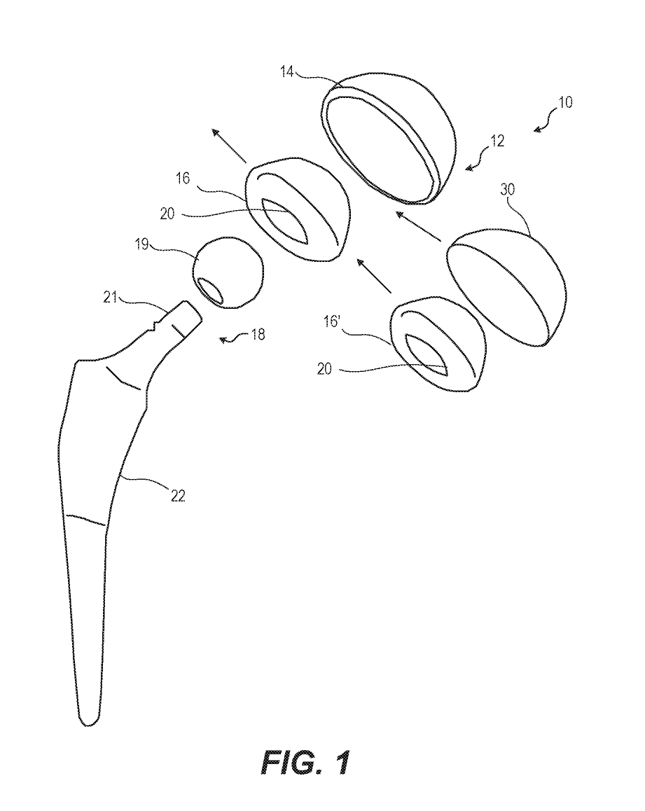

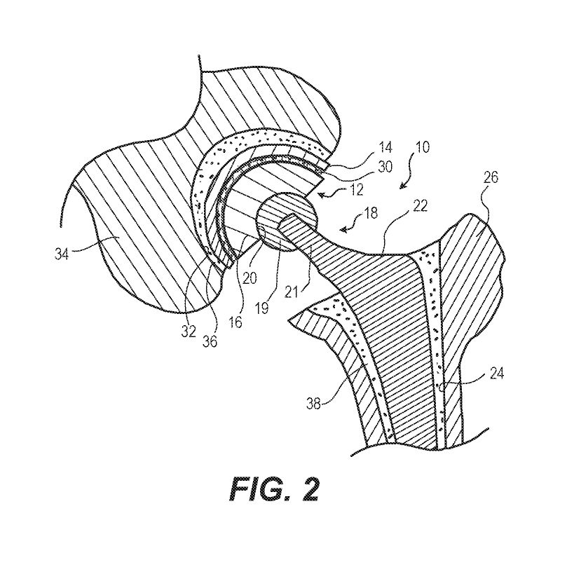

FIGS. 1-2 depict a prosthetic hip assembly, in particular a dual mobility femoral head construct 10, for replacing a human hip joint. An acetabular liner assembly 12 as originally implanted includes a secure acetabular shell 14 of a porous metal that fuses with the pelvic bone and an articular head insert 16 received for rotation therein. A femoral prosthesis 18 that is spherically shaped is received for articulating movement within a hemispherical recess 20 in the articular head insert 16. The femoral prosthesis 18 replaces the natural femoral head. The femoral prosthesis 18 includes an articulating head component 19 is assembled to distal canted end 21 of a femoral stem component 22 that is seated in a prepared intramedullary space 24 of a femur 26.

With particular reference to FIG. 1, the original articular head insert 16 that is failed is removed and replaced with an inner acetabular liner 30 sized for the inner diameter (ID) of the secure acetabular shell 14. The latter includes features discussed below to facilitate a long-lasting cement attachment to the secure acetabular shell 14. The inner acetabular liner 30 receives for movement an appropriately sized articular head insert 16′. The original acetabular shell 14, such as composed of Titanium or Tantalum, has typically not failed and is left in place. The replacement articular head insert 16′ is generally made of biocompatible polymer such as polyethylene, polyether ether ketone (PEEK), polyaryletherketone (“PAEK”), an ultra-high molecular weight polyethylene polymer material (“UHMWPE”) such as an antioxidant stabilized UHMWPE or another equivalent plastic material. In other exemplary embodiments, the biocompatible polymer may be a polyolefin, polyester, polyimide, polyamide, polyacrylate, and/or other suitable polymers.

With continued reference to FIGS. 1-2, the acetabular liner assembly 12 lines an acetabulum 32 on the pelvic side of the hip joint. The acetabular liner assembly 12 is pressed into the prepared acetabulum 32 of a pelvis 34. The secure acetabular shell 14 may abut the bone or a layer of bone cement 36 may be positioned within the acetabulum 32 and the secure acetabular shell 14 to lock the acetabular liner assembly 12 in place. The femoral stem component 22 may abut bone of the femur 26 or a layer of bone cement 38 may be positioned between the bone and the femoral stem component 22. acetabular liner assembly

The articulating head component 19 may be permanently affixed to the femoral stem component 22 or it may be a modular piece fit on the femoral stem component 22 at the time of surgery. After the acetabular liner assembly 12 and femoral stem component 22 has been implanted, the articulating head component 19 is inserted into the concave bearing surface of the hemispherical recess 20 of the articular head insert 16 to restore normal hip joint function. In one embodiment, the articulating head component 19 is produced from a biocompatible metal, e.g. a titanium alloy, a cobalt-chromium alloy, or a stainless steel alloy and may be coated by a hard, low friction coating such as amorphous diamond-like coating (ADLC). Alternatively, the head may be produced from carbon, e.g. a pyrolytic carbon, with essentially the same surface characteristics as ADLC.

By way of example but not limitation, the articulating head component 19 may comprise of cobalt chrome, titanium, titanium alloys, tantalum, tantalum alloys or other metals and/or metal alloys consistent with the present invention. Among other things, the articulating head component 19 is preferably a material, which is highly biocompatible. By way of further example but not limitation the articulating head component 19 may comprise CoCrMo, cobalt based alloys, stainless steels, zirconium based alloys or other metals and/or metal alloys consistent with the present invention. In one embodiment, the articulating head component 19 is preferably a material which has relatively high hardness and which is scratch resistant. For the purposes of the present invention, the term bimetal may be defined as a composite of two metals, where each of the metals has a different primary constituent. The bimetal construct can be formed from two different commercially pure metals, from two alloys of different base metals, or a combination thereof.

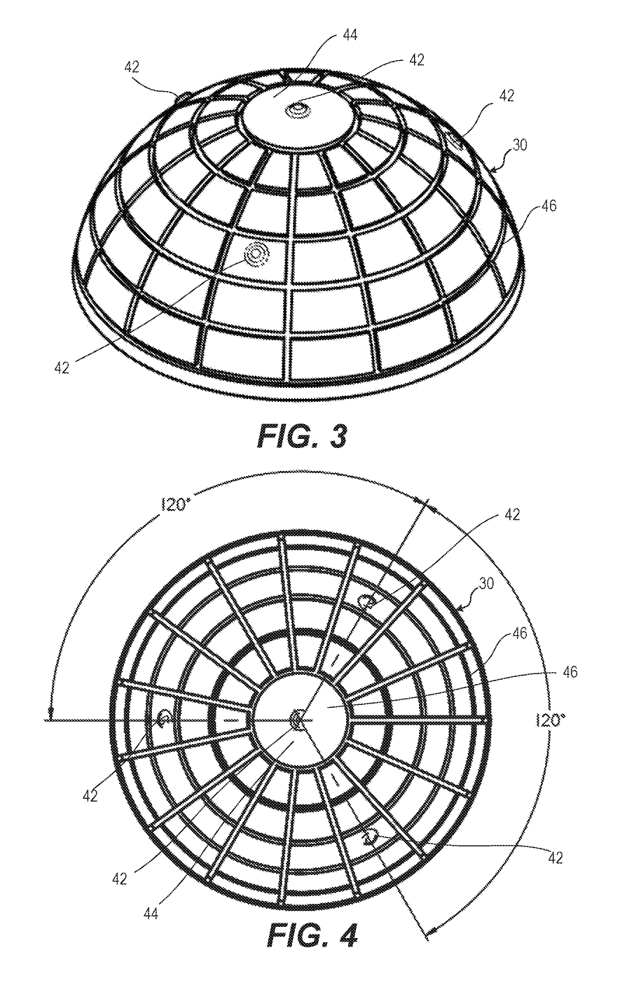

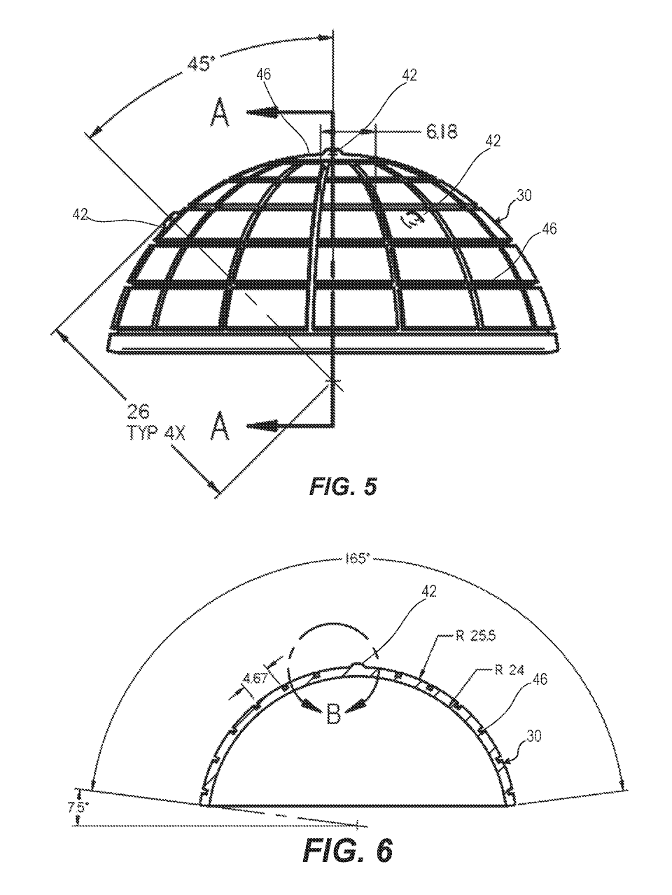

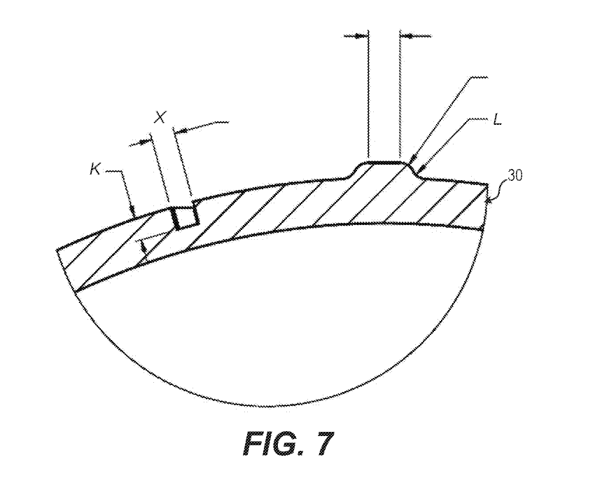

With reference to FIGS. 3-7, the new inner acetabular liner 30 is depicted in various orientations and details to depict features for facilitating fixation of inner acetabular liner 30 to the secure acetabular shell 14. The connection between inner acetabular liner 30 to the secure acetabular shell 14 could be a direct connection with the contacting surface, or an indirect connection with the contacting surface. In the embodiments where the connection is an indirect connection, a material could be positioned between the new inner acetabular liner 30 and the secure acetabular shell 14. The material could be a material selected from a group consisting of: adhesive materials, elastic materials and bone cement. The material could be at least one of: bone cement, an at least partly elastic material, glue, adhesive, antibiotic, biocompatible plastic material, biocompatible ceramics or biocompatible metal.

However it is also conceivable that the connection is assisted or replaced with at least one screw, at least one pin, at least one portion of at least one of the parts adapted to be introduced into the other part, the parts being adapted to be sliding into the other part, form fitting, welding, adhesive, pin, wire, a ball mounted into portions of the parts, a male portion of one part mounted into a female portion of the other part, a key introduced into a lock portion of parts, band, or other mechanical connecting members. The new inner acetabular liner 30 can have surface features such as textures, grooves, knurling, etc.

In some embodiments, the new inner acetabular liner 30 can be held in place to the secure acetabular shell 14 by a friction fit. In some embodiments, a retention material, such as adhesives can be used to hold the new inner acetabular liner 30 in place to the secure acetabular shell 14. In other embodiments, the new inner acetabular liner 30 can have a mating feature that couples with a complementary mating feature on the secure acetabular shell 14, such as hooks, splines, tabs, channels and grooves, or any other mating features as would be known in the art.

In one embodiment, the inner acetabular liner 30 is attached using an adhesive. In one embodiment, the adhesive is a biocompatible adhesive such polymethylmethacrylate (PMMA) or other non-resorbable cement. The cement is generally in liquid or paste form but may be a powder or solid to which a liquid component is added.

In one embodiment, the new inner acetabular liner 30 is a cementable liner comprising metal, ceramic, polymer or combinations thereof. In one embodiment, the new inner acetabular liner 30 is formed from a medical-grade metal such as stainless steel, cobalt chrome, or titanium, although other metals or alloys may be used. Moreover, in some embodiments, rigid polymers such as polyetheretherketone (PEEK) may also be used. In one embodiment, titanium alloy or cobalt chromium alloy may be used. However, if the components, and in particular the new inner acetabular liner 30, are more modular in nature, then different materials can be considered to improve workability, cost-savings, implantation, and ultimately use by the patient once implanted. In one embodiment, the new inner acetabular liner 30 is formed from polyethylene.

In one embodiment, the inner acetabular liner 30 is formed from cast a Cobalt-Chromium (CoCr) alloy. For example, the inner acetabular liner 30 may have a highly polished Inner Diameter (ID) starting at 36 mm ID and 40 outer diameters (OD). Using cast CoCr alloy allows for a very thin liner, optimizing the ID of the liner, the size of the dual mobility head, and stability of the hip. The polished ID optimizes wear characteristics between the inner acetabular liner 30 and the articular head insert 16. The cast technique also allows for a higher elasticity than a forged CoCr liner, which may optimize liner/cement interface stability.

Second, with particular reference to FIGS. 3-5, polymethyl methacrylate (PMMA) spacers 42 marked at the apex of the OD and on each side of the inner acetabular liner 30. For example, three PMMA spacers 42 may be annularly spaced 120° a part at the exact mid-point of radius of curvature in addition to the apex. It should be appreciated that PMMA material is illustrative and that other materials may be used. These PMMA spacers 42 allow a uniform cement mantle of at least 0.5 mm between the secure acetabular shell 14 and apex 44 of the CoCr OD of the inner acetabular liner 30 and 1.25 mm of cement interdigitation within the longitudinal and circumferential and longitudinally “depressed” spider webbing 46. Uniformity of cement mantle is important for mechanical stability.

Third, the spider webbing 46 has a 0.75 mm depressed design with 0.5 mm pole to optimize lever out and torsional forces by cement/liner interdigitation and stability.

Fourth, with particular reference to FIG. 2 and, a truncated 165° design stops short of a true 180° hemispherical design to avoids neck impingement on the inner acetabular liner 30. Older designed stem tended to have larger necks creating earlier impingement during Range of Motion (ROM). When using this product in a revision setting avoids early impingement and potential dislocation of the hip.

In the known devices, a distinction can be made between single-mobility head/liner constructs, dual-mobility head/liner constructs, within or excluding modular acetabular systems. In the single-mobility head/liner constructs, the polyethylene or ceramic insert is fixed in an insertion cup and has a coaxial and substantially hemispherical articular cavity permitting the engagement and pivoting of the spherical head of the first part of the prosthesis. The rotation movements of the joint then take place between the spherical head of the first part of the prosthesis and the articular cavity of the articular insert fixated within a modular acetabular shell system. In a dual-mobility head/liner construct, the articular insert is itself mounted rotatably in the insertion cup, thereby providing a first sliding surface between the insertion cup and the articular insert, and a second sliding surface between the articular insert and the spherical head. This head articulates within a non-modular acetabular shell, or a modular acetabular shell with a liner. In the acetabulums with a non-modular acetabular system, the articular insert has a spherical outer surface so as to be rotatably mounted directly in the cotyloid cavity of the pelvis of a patient. Alternatively, a modular acetabular system has a spherical outers surface so as to be rotatably mounted directly in the cotyloid cavity of the pelvis of a patient, and receive a fixate modular articulating surface comprised of numerous bearing surface material options.

Thus, by virtue of the foregoing, in one aspect the present disclosure provides an implantable prosthetic device having an inner hemispherical liner formed from a cast cobalt-chromium alloy and having an outer diameter sized for attachment inside a secure acetabular shell implanted into an acetabular recess in a pelvis. At least three spacers are annularly displaced about an outer diameter of the inner hemispherical liner to define a uniform cement thickness with the secure acetabular shell. Web shaped depressions are formed circumferentially and longitudinally in the outer diameter of the inner hemispherical liner to receive cement.

In another aspect, the present disclosure provides an implantable prosthetic assembly having a secure acetabular shell received within an acetabular recess formed in a pelvis. An implantable prosthetic device has an inner hemispherical liner formed from a cast cobalt-chromium alloy and sized for attachment inside the secure acetabular shell. At least three spacers annularly are displaced about an outer diameter of the inner hemispherical liner to define a uniform cement thickness with the secure acetabular shell. Web shaped depressions are formed circumferentially and longitudinally in the outer diameter of the inner hemispherical liner to receive cement. An articular head insert is received for rotational movement in an inner diameter of the inner hemispherical liner. A femoral head implant is received for articulating movement in the articular head insert.

In one or more embodiments, an implantable prosthetic device can have at least three spacers comprise polymethyl methacrylate (PMMA). For example, the at least three spacers comprise a first spacer can be attached to an apex of the hemispherical liner and at least three spacers annularly spaced at a midpoint of a radius of curvature of the outer diameter. The at least three spacers can extend 0.5 mm from the outer diameter. The web shaped depressions can be 0.75 mm deep. The hemispherical liner can include a truncated radius of curvature limited to 165° with respect to a center of articulating movement of a femoral head received in an articular head insert received in turn for dual mobility by the hemispherical liner.

In one or more embodiments, an implantable prosthetic assembly can include a secure acetabular shell received within an acetabular recess formed in a pelvis. An implantable prosthetic device can include (i) a hemispherical liner formed from a cast cobalt-chromium alloy, (ii) at least three spacers annularly displaced about an outer diameter of the hemispherical liner to define a uniform cement thickness with the secure acetabular shell, and (iii) web shaped depressions formed circumferentially and longitudinally in the outer diameter of the hemispherical liner to receive cement. An articular head insert is received for rotational movement in an inner diameter of the hemispherical liner. A femoral head implant is received for articulating movement in the articular head insert.

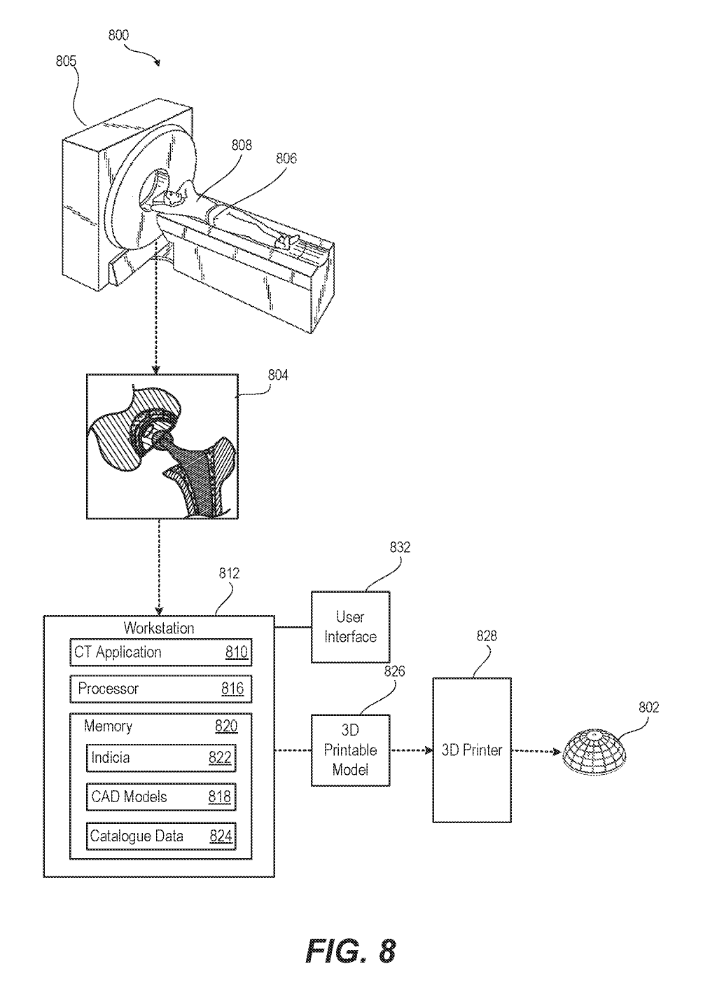

CUSTOM MATCHED JOINT PROSTHESIS REPLACEMENT. FIG. 8 illustrates a system 800 that creates a matching replacement prosthesis component 802 such as an acetabular shell based on a medical diagnostic computed tomography (CT) scan 804 made by a CT diagnostic system 805 of a currently implanted component 806 in a patient 808. An analysis engine, such an industrial CT application 810 executed on a controller, such as a workstation 812, creates a 3D model 814. The industrial CT application 810 can perform one or more processes executed by a processor 816 to provide identifying information about the implanted component 806. For example, the industrial CT application 810 can perform part-to-CAD (computer aided design) comparisons compared to CAD models 818 of known designs contained in memory 820. In some instances, medical records may indicate the type of the currently implanted component 806, allowing for searching for the appropriate CAD model 818 that was created by the original equipment manufacturer (OEM). For another example, the industrial CT application 810 can locate indicia 822 of a source and type imprinted onto the implanted component 806. For an additional example, For example, the industrial CT application 810 can perform geometric dimensioning and tolerance (GD&T) analysis to use in looking up catalogue data 824 of a corresponding component. If a match is found, a 3D printable model 826 can be provided to a 3D printer 828 to create the matching prosthesis component 802. If a specific match is not found, a default design 830 that approximates the required dimensions can be located. For a further example, the GD&T 802 can convert the 3D model into 3D printable model 826 suitable for 3D printing by a 3D printer 828 and to serve as the starting point for three-dimensional (3D) printing of a corresponding replacement prosthesis component 802. The industrial CT application Bio can dimensionally verify any male protrusions and holes that are supposed to mate respectively between currently implanted component 806 and the matching replacement prosthesis component 802. An operator could customize an existing model, default model, or scan-to-part model to accommodate irregularities in the currently implanted component 806 via a user interface 832. Additionally, the system 800, based upon knowledge of the old prosthesis (currently implanted component 806), could make additions to the design of the shell that would conform to the prosthesis, such as holes in the cup or surface features of the cup. This for example could include an apical peg or offset peg that in matching replacement prosthesis component 802 would allow the surgeon to insert the shell into the cup with the peg (male) inserting into the hole of the cup for rotational or translational stability.

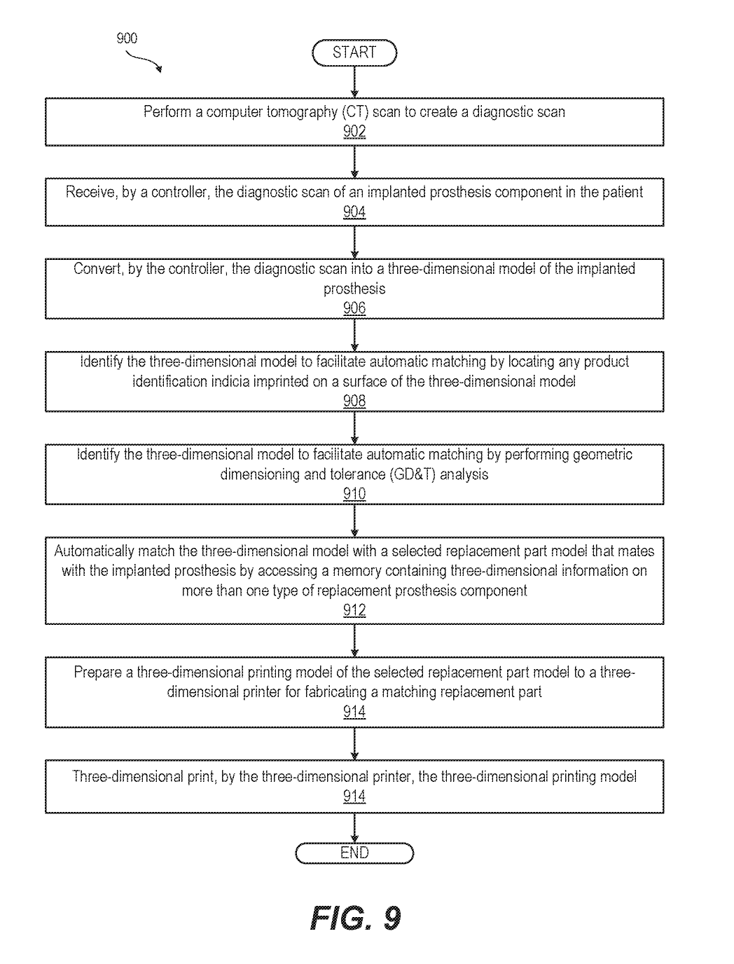

FIG. 9 illustrates a method 900 of fabricating a replacement prosthesis component for implantation into a patient. In one or more embodiments, the method 900 includes performing a computer tomography (CT) scan to create a diagnostic scan (block 902). The method 900 includes receiving, by a controller, the diagnostic scan of an implanted prosthesis component in the patient (block 904). The method 900 includes converting, by the controller, the diagnostic scan into a three-dimensional model of the implanted prosthesis (block 906). The method 900 includes identifying the three-dimensional model to facilitate automatic matching by locating any product identification indicia imprinted on a surface of the three-dimensional model (block 908). The method 900 includes identifying the three-dimensional model to facilitate automatic matching by performing geometric dimensioning and tolerance (GD&T) analysis (block 910). The method 900 includes automatically matching the three-dimensional model with a selected replacement part model that mates with the implanted prosthesis by accessing a memory containing three-dimensional information on more than one type of replacement prosthesis component (block 912). The method goo includes preparing a three-dimensional printing model of the selected replacement part model to a three-dimensional printer for fabricating a matching replacement part (block 914). The method 900 includes three-dimensional printing, by the three-dimensional printer, the three-dimensional printing model (block 916).

In accordance with various aspects of the disclosure, an element, or any portion of an element, or any combination of elements may be implemented with a “processing system” that includes one or more physical devices comprising processors. Non-limiting examples of processors include microprocessors, microcontrollers, digital signal processors (DSPs), field programmable gate arrays (FPGAs), programmable logic devices (PLDs), programmable logic controllers (PLCs), state machines, gated logic, discrete hardware circuits, and other suitable hardware configured to perform the various functionality described throughout this disclosure. One or more processors in the processing system may execute instructions. A processing system that executes instructions to effect a result is a processing system which is configured to perform tasks causing the result, such as by providing instructions to one or more components of the processing system which would cause those components to perform acts which, either on their own or in combination with other acts performed by other components of the processing system would cause the result. Software shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise. The software may reside on a computer-readable medium. The computer-readable medium may be a non-transitory computer-readable medium. Computer-readable medium includes, by way of example, a magnetic storage device (e.g., hard disk, floppy disk, magnetic strip), an optical disk (e.g., compact disk (CD), digital versatile disk (DVD)), a smart card, a flash memory device (e.g., card, stick, key drive), random access memory (RAM), read only memory (ROM), programmable ROM (PROM), erasable PROM (EPROM), electrically erasable PROM (EEPROM), a register, a removable disk, and any other suitable medium for storing software and/or instructions that may be accessed and read by a computer. The computer-readable medium may be resident in the processing system, external to the processing system, or distributed across multiple entities including the processing system. The computer-readable medium may be embodied in a computer-program product. By way of example, a computer-program product may include a computer-readable medium in packaging materials. Those skilled in the art will recognize how best to implement the described functionality presented throughout this disclosure depending on the particular application and the overall design constraints imposed on the overall system.

“Processor” means devices, which can be configured to perform the various functionality set forth in this disclosure, either individually or in combination with other devices. Examples of “processors” include microprocessors, microcontrollers, digital signal processors (DSPs), field programmable gate arrays (FPGAs), programmable logic devices (PLDs), programmable logic controllers (PLCs), state machines, gated logic, and discrete hardware circuits. The phrase “processing system” is used to refer to one or more processors, which may be included in a single device, or distributed among multiple physical devices.

“Instructions” means data, which can be used to specify physical or logical operations, which can be performed by a processor. Instructions should be interpreted broadly to include, code, code segments, program code, programs, subprograms, software modules, applications, software applications, software packages, routines, subroutines, objects, executables, threads of execution, procedures, functions, hardware description language, middleware, etc., whether encoded in software, firmware, hardware, microcode, or otherwise.

The various embodiments may be implemented in any of a variety of computing devices. A computing device will typically include a processor coupled to volatile memory and a large capacity nonvolatile memory, such as a disk drive of Flash memory. The computing device may also include a floppy disc drive and a compact disc (CD) drive coupled to the processor. The computing device may also include a number of connector ports coupled to the processor for establishing data connections or receiving external memory devices, such as a USB or FireWire™ connector sockets, or other network connection circuits for establishing network interface connections from the processor to a network or bus, such as a local area network coupled to other computers and servers, the Internet, the public switched telephone network, and/or a cellular data network. The computing device may also include the trackball or touch pad, keyboard, and display all coupled to the processor.

The various embodiments may also be implemented on any of a variety of commercially available server devices, such as the server. Such a server typically includes a processor coupled to volatile memory and a large capacity nonvolatile memory, such as a disk drive. The server may also include a floppy disc drive, compact disc (CD) or DVD disc drive coupled to the processor. The server may also include network access ports coupled to the processor for establishing network interface connections with a network, such as a local area network coupled to other computers and servers, the Internet, the public switched telephone network, and/or a cellular data network.

All publications, patents and patent applications cited herein, whether supra or infra, are hereby incorporated by reference in their entirety to the same extent as if each individual publication, patent or patent application was specifically and individually indicated as incorporated by reference. It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein, will only be incorporated to the extent that no conflict arises between that incorporated material and the existing disclosure material.

It must be noted that, as used in this specification and the appended claims, the singular forms “a,” “an” and “the” include plural referents unless the content clearly dictates otherwise. Thus, for example, reference to a “colorant agent” includes two or more such agents.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

As will be appreciated by one having ordinary skill in the art, the methods and compositions of the invention substantially reduce or eliminate the disadvantages and drawbacks associated with prior art methods and compositions.

It should be noted that, when employed in the present disclosure, the terms “comprises,” “comprising,” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

While it is apparent that the illustrative embodiments of the invention herein disclosed fulfill the objectives stated above, it will be appreciated that numerous modifications and other embodiments may be devised by one of ordinary skill in the art. Accordingly, it will be understood that the appended claims are intended to cover all such modifications and embodiments, which come within the spirit and scope of the present invention.

What is claimed is:

1. A method of fabricating a replacement prosthesis component for implantation into a patient, the method comprising:
receiving a diagnostic scan of an implanted prosthesis component in the patient;
converting the diagnostic scan into a three-dimensional model of the implanted prosthesis;
automatically matching the three-dimensional model with a selected replacement part model that mates with the implanted prosthesis; and
preparing a three-dimensional printing model of the selected replacement part model to a three-dimensional printer for fabricating a matching replacement part.

2. The method of claim 1, further comprising performing a computer tomography (CT) scan to create the diagnostic scan.

3. The method of claim 1, further comprising identifying the three-dimensional model to facilitate automatic matching by locating product identification indicia imprinted on a surface of the three-dimensional model.

4. The method of claim 1, further comprising identifying the three-dimensional model to facilitate automatic matching by performing geometric dimensioning and tolerance (GD&T) analysis.

5. The method of claim 1, further comprising three-dimensional printing, by the three-dimensional printer, the three-dimensional printing model.

6. The method of claim 1, wherein:
the implanted prosthesis component comprises a secure acetabular shell implanted into a acetabular recess in a pelvis; and
the replacement prosthesis component comprises a hemispherical liner formed from a cast cobalt-chromium alloy and having an outer diameter sized for attachment inside the secure acetabular shell.

7. The method of claim 6, wherein the hemispherical liner comprises:
at least three spacers annularly displaced about an outer diameter of the hemispherical liner to define a uniform cement thickness with the secure acetabular shell; and
web shaped depressions formed circumferentially and longitudinally in the outer diameter of the hemispherical liner to receive cement.

8. The method of claim 7, wherein:
the at least three spacers comprise polymethyl methacrylate (PMMA); and
the at least three spacers comprise a first spacer attached to an apex of the hemispherical liner and at least three spacers annularly spaced at a midpoint of a radius of curvature of the outer diameter.

9. The method of claim 7, wherein:
the at least three spacers extend 0.5 mm from the outer diameter; and
the web shaped depressions are 0.75 mm deep.

10. The method of claim 6, wherein the hemispherical liner comprises a truncated radius of curvature limited to 165° with respect to a center of articulating movement of a femoral head received in an articular head insert received in turn for dual mobility by the hemispherical liner.

11. An apparatus of fabricating a replacement prosthesis component for implantation into a patient, the apparatus comprising:
a memory containing three-dimensional information on more than one type of replacement prosthesis component;
a controller communicatively coupled to the memory and a three-dimensional printer, the controller:
receives a diagnostic scan of an implanted prosthesis component in the patient;
converts the diagnostic scan into a three-dimensional model of the implanted prosthesis;
automatically matches the three-dimensional model with a selected replacement part model that mates with the implanted prosthesis; and
prepares a three-dimensional printing model of the selected replacement part model to a three-dimensional printer for fabricating a matching replacement part.

12. The apparatus of claim 11, further comprising a CT scanner communicatively coupled to the controller to perform a computer tomography (CT) scan to create the diagnostic scan.

13. The apparatus of claim 11, wherein the controller identifies the three-dimensional model to facilitate automatic matching by locating product identification indicia imprinted on a surface of the three-dimensional model.

14. The apparatus of claim 11, wherein the controller identifies the three-dimensional model to facilitate automatic matching by performing geometric dimensioning and tolerance (GD&T) analysis.

15. The apparatus of claim 11, further comprising the three-dimensional printer to receive three-dimensional printing model and to fabricate the selected replacement prosthesis component.

16. The apparatus of claim 11, wherein:
the implanted prosthesis component comprises a secure acetabular shell implanted into a acetabular recess in a pelvis; and
the replacement prosthesis component comprises a hemispherical liner formed from a cast cobalt-chromium alloy and having an outer diameter sized for attachment inside the secure acetabular shell.

17. The apparatus of claim 16, wherein the hemispherical liner comprises:
at least three spacers annularly displaced about an outer diameter of the hemispherical liner to define a uniform cement thickness with the secure acetabular shell; and
web shaped depressions formed circumferentially and longitudinally in the outer diameter of the hemispherical liner to receive cement.

18. The apparatus of claim 17, wherein:
the at least three spacers comprise polymethyl methacrylate (PMMA); and
the at least three spacers comprise a first spacer attached to an apex of the hemispherical liner and at least three spacers annularly spaced at a midpoint of a radius of curvature of the outer diameter.

19. The apparatus of claim 17, wherein:
the at least three spacers extend 0.5 mm from the outer diameter; and
the web shaped depressions are 0.75 mm deep.

20. The apparatus of claim 16, wherein the hemispherical liner comprises a truncated radius of curvature limited to 165° with respect to a center of articulating movement of a femoral head received in an articular head insert received in turn for dual mobility by the hemispherical liner.
